(19) United States
(12) Patent Application Publication
(10) Pub. No.: US 2025/0229056 A1
(43) Pub. Date: Jul. 17, 2025

(54) APPARATUS AND METHOD FOR CLEARING A CATHETER BLOCKAGE FLUSHING AN ORGAN, OR INSTILLING MEDICINE INTO AN ORGAN

(71) Applicant: Kumrija Son, Lakeville, MN (US)

(72) Inventor: Kumrija Son, Lakeville, MN (US)

(21) Appl. No.: 19/018,822

(22) Filed: Jan. 13, 2025

Related U.S. Application Data
(60) Provisional application No. 63/621,077, filed on Jan. 15, 2024.

(51) Int. Cl. A61M 25/00; A61M 25/10; A61M 39/10; A61M 39/12

(52) U.S. Cl. CPC A61M 25/0017; A61M 25/0026; A61M 39/12; A61M 2025/0019; A61M 2025/1061; A61M 2039/1083; A61M 2210/1085

(57) ABSTRACT

An apparatus and a corresponding method for flushing fluid through a catheter that has, or that is connected to tubing that has, a Luer lock and a Luer-lock interface, wherein the Luer lock opens when a Luer-lock connector is connected to the Luer-lock interface. In some embodiments, the system includes a monolithic structure that includes a fluid-source connector configured to removably connect to a fluid source; a Luer-lock connector configured to operatively connect to the Luer-lock interface on the catheter; and a fluid passageway between the fluid-source connector and the Luer-lock connector. Some embodiments are used for irrigating and flushing an organ (such as flushing an organ with a medication, cancer-treatment fluid, or the like, or instilling medication, cancer-treatment fluid, or the like into the organ).

### CROSS-REFERENCE TO RELATED APPLICATION

This application claims priority benefit, under 35 U.S.C. § 119(e), of U.S. Provisional Patent Application No. 63/621,077, filed Jan. 15, 2024 by Kumrija Son and titled “Apparatus and method for clearing a catheter blockage,” which is incorporated herein by reference in its entirety.

### FIELD OF THE INVENTIONS

This invention relates to the field of medical apparatus and procedures, and more specifically to a method and apparatus for connecting a squeezable source of sterile saline, such as a conventional resilient or flexible saline bottle or saline bag, to a Luer-lock connection on a catheter in order to force saline and/or medicament(s) from the resilient or flexible saline bottle or saline bag into the catheter to dislodge a blockage, maintain patency, and/or irrigate a patient's internal organ (such as the patient's bladder), wherein the invention is usable with any of a variety of catheter types such as indwelling urinary catheters, enteric catheters, and the like.

### BACKGROUND OF THE INVENTION

Often in the nursing field, it is necessary force liquids into indwelling catheters in order to remove blockages. Sometimes, it is also necessary to irrigate maintained tubes, even if there are no obstructions. In such cases, the attending medical staff may have orders to irrigate tubes every four hours, for example in the cases of nephrostomy tubes, urostomy tubes, or fecal tubes, not just to solve the obstruction problem, but also to maintain consistent work flow and prevent future problems.

Foley catheters are the most common type of indwelling urinary catheter. A Foley catheter (sometimes called a “two-way Foley catheter” or “balloon catheter”) includes two separate parallel channels (called lumens): a first (drain) lumen for draining urine, and a second (inflation) lumen for inflating a distal balloon tip with sterile water to keep the distal tip of the Foley catheter in place in a patient's bladder.

Three-way Foley catheters function like regular two-way Foley catheters except that three-way Foley catheters have a third lumen used to convey a sterile solution into the bladder to irrigate the inside of the bladder, and that sterile solution is then flushed out through the drain lumen of the three-way Foley catheter. People who are prone to develop clots or sediment might use three-way Foley catheters, however there may still be a need to occasionally back-flush the drain lumen of a three-way Foley catheter to remove sediment blockages.

People who have mobility issues, such as quadriplegia, may use Foley catheters. Foley catheters are usually used for up to a month at a time, and carry a higher risk of blockages of the drain lumen and a higher risk of infection than other types of catheters, such a single-use or intermittent catheters. Foley catheters are typically prescribed to people who cannot use another type catheter on their own.

U.S. Pat. No. 8,747,372 issued Jun. 10, 2014 to Joseph P. Schultz with the title “Abscess Irrigation Systems”, and is incorporated herein by reference. U.S. Pat. No. 8,747,372 describes irrigation systems having splash shields attachable to bottles or syringes, also including abscess irrigation systems. U.S. Pat. No. 8,747,372 is a continuation-in-part of application Ser. No. 11/753,560, filed May 24, 2007, titled “Splash shield systems”, which is related to and claim priority from prior provisional application Ser. No. 60/803,109, filed May 24, 2006, titled “Splash Shield System”, and which application Ser. No. 11/753,560, is a continuation-in-part of application Ser. No. 11/317,758, filed Dec. 23, 2005, titled “Splash shield system”, which is a continuation-in-part of application Ser. No. 10/245,241, filed Sep. 17, 2002, titled “Splash shield system”, which is a continuation-in-part of application Ser. No. 10/123,966, filed Apr. 16, 2002, titled “Medical component system”, which is a continuation-in-part of application Ser. No. 09/484,666, filed Jan. 18, 2000, titled “Medical component system”, each of which is incorporated herein by reference.

U.S. Pat. No. 11,083,835 issued Aug. 10, 2021 to Joseph P. Schultz with the title “Abscess Irrigation Systems”, and is incorporated herein by reference. U.S. Pat. No. 11,083,835 describes an irrigator system for delivering a volume of fluid from a fluid source to an abscess or wound. The irrigator system includes a fluid source connector for removably connecting to the fluid source, a fluid delivery nozzle for delivering the fluid to the abscess or wound, and a shield removably positioned around the fluid delivery nozzle to protect the fluid delivery nozzle. U.S. Pat. No. 11,083,835 claims priority from, as a continuation application, U.S. patent application Ser. No. 16/197,480, filed on Nov. 21, 2018, now U.S. Pat. No. 10,576,619, which is a continuation application of U.S. patent application Ser. No. 14/296,651, filed on Jun. 5, 2014, now U.S. Pat. No. 10,363,356, which is a continuation application of U.S. patent application Ser. No. 12/851,476, filed on Aug. 5, 2010, now U.S. Pat. No. 8,747,372 (set forth above), which claims the benefit of U.S. Provisional Application No. 61/325,756, filed Apr. 19, 2010, and U.S. Provisional Application No. 61/231,638, filed Aug. 5, 2009, each of which is incorporated herein by reference.

There remains a need for an improved, easy-to-use system and apparatus, readily usable in exigent circumstances, to quickly connect conventional saline bottles or saline bags to indwelling catheters to remove blockages, and/or to irrigate internal organs with saline and/or medicaments via maintained tubes, even if there are no obstructions, in order to prevent future problems.

### SUMMARY OF THE INVENTION

The present invention provides a system for flushing a catheter that has a Luer-lock interface. This system includes a monolithic structure that includes: a fluid-source connector configured to removably connect to a fluid source; a Luer-lock connector configured to operatively connect to the Luer lock interface on the catheter; and a fluid passageway between the fluid-source connector and the Luer-lock connector. In some embodiments of the system, the fluid-source connector and the Luer-lock connector are integrally formed from a unitary rigid piece of polymer. In some embodiments of the system, the fluid-source connector includes a saline-bag spike. In some embodiments of the system, the fluid-source connector includes a female threaded saline-bottle connector. In some embodiments of the system, the fluid-source connector includes both a saline-bag spike and a female threaded saline-bottle connector that surrounds at least a portion of the saline-bag spike.

In some embodiments of the system, the fluid source contains sterile saline and/or one or more medicaments.

Some embodiments are used for irrigating and flushing an organ (such as flushing an organ with a medication, cancer-treatment fluid, or the like, or instilling medication, cancer-treatment fluid, or the like into the organ).

In some embodiments of the system, the catheter is an indwelling Foley catheter configured to drain urine from a patient's bladder and/or irrigate the bladder.

### BRIEF DESCRIPTION OF THE DRAWINGS

FIG. 1A is a cross-section view of a fluid interface 101 that includes a Luer-lock connector 110, a threaded bottle connector 120 and a spike connector 130, along with a removable protective spike cover 140 and a removable protective Luer cover 150, according to some embodiments of the present invention.

FIG. 1B is a side view of another fluid interface 102, according to some embodiments of the present invention.

FIG. 1C is a cross-section view of fluid interface 102 that includes a Luer-lock connector 110, a threaded bottle connector 120 and a spike connector 130, for use with a removable protective spike cover 140 (as shown in FIG. 1A) and a removable protective Luer cover 150 (as shown in FIG. 1A), according to some embodiments of the present invention.

FIG. 2A is a cross-section view of a fluid interface 201 that includes a Luer-lock connector 210, a threaded bottle connector 220, along with a removable protective Luer cover 250, according to some embodiments of the present invention.

FIG. 2B is a side view of another fluid interface 202, according to some embodiments of the present invention.

FIG. 2C is a cross-section view of fluid interface 202 that includes a Luer-lock connector 210 and a threaded bottle connector 220, according to some embodiments of the present invention.

FIG. 3A is a front view of a fluid interface 301.

FIG. 3B is a top view of fluid interface 301.

FIG. 3C is a left-side view of fluid interface 301.

FIG. 3D is a back view of fluid interface 301.

FIG. 3E is a right-side view of fluid interface 301.

FIG. 3F is a bottom view of fluid interface 301.

FIG. 3G is an upper isometric view of fluid interface 301.

FIG. 3H is a lower isometric view of fluid interface 301.

FIG. 3I is a lower isometric view of fluid interface 301 showing internal structures in dashed lines.

FIG. 3J is a cross-section view of fluid interface 301 that includes a Luer-lock connector 310, a middle section 320 and a spike connector 330, along with a removable protective spike cover 340 and a removable protective Luer cover 350, according to some embodiments of the present invention.

FIG. 3K is a cross-section view of another fluid interface 302 that includes a Luer-lock connector 310, a middle section 329 of arbitrary length and a spike connector 339 of arbitrary length, according to some embodiments of the present invention.

FIG. 4A is a front view of a fluid interface 401.

FIG. 4B is a left-side view of fluid interface 401.

FIG. 4C is a top view of fluid interface 401.

FIG. 4D is a right-side view of fluid interface 401.

FIG. 4E is a back view of fluid interface 401.

FIG. 4F is an upper isometric view of fluid interface 401.

FIG. 4G is a lower isometric view of fluid interface 401.

FIG. 4H is a cross-section view of fluid interface 401.

FIG. 4I is another upper isometric view of fluid interface 401.

FIG. 4J another lower isometric view of fluid interface 401.

FIG. 5A is a right-side view of a fluid interface 501.

FIG. 5B is a top view of fluid interface 501.

FIG. 5C is a front view of fluid interface 501.

FIG. 5D is a left-side view of fluid interface 501.

FIG. 5E is a back view of fluid interface 501.

FIG. 5F is a bottom view of fluid interface 501.

FIG. 5G is an upper isometric view of fluid interface 501.

FIG. 5H is a cross-section view of fluid interface 501 that includes a Luer-lock connector 510, a threaded bottle connector 520 having rounded outer edges, and a slotted spike connector 520, according to some embodiments of the present invention.

FIG. 5I is an upper isometric view of fluid interface 501.

FIG. 5J is a lower isometric view of fluid interface 501.

FIG. 6A is a right-side view of a fluid interface 601.

FIG. 6B is a top view of fluid interface 601.

FIG. 6C is a front view of fluid interface 601.

FIG. 6D is a left-side view of fluid interface 601.

FIG. 6E is a back view of fluid interface 601.

FIG. 6F is a bottom view of fluid interface 601.

FIG. 6G is an upper isometric view of fluid interface 601.

FIG. 6H is a lower isometric view of fluid interface 601.

FIG. 6I is another lower isometric view of fluid interface 601.

FIG. 6J is a cross-section view of another fluid interface 602 that includes a Luer-lock connector 610, a threaded cap (bottle connector) 620 and a spike connector 630, according to some embodiments of the present invention.

FIG. 6K is a side view of fluid interface 601 showing internal structures in dashed lines.

FIG. 7 is a block diagram side view of a system 700 for flushing catheter blockages and/or irrigating a patient's bladder.

### DESCRIPTION OF PREFERRED EMBODIMENTS

Although the following detailed description contains many specifics for the purpose of illustration, a person of ordinary skill in the art will appreciate that many variations and alterations to the following details are within the scope of the invention. Specific examples are used to illustrate particular embodiments; however, the invention described in the claims is not intended to be limited to only these examples, but rather includes the full scope of the attached claims. Accordingly, the following preferred embodiments of the invention are set forth without any loss of generality to, and without imposing limitations upon the claimed invention. Further, in the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The embodiments shown in the Figures and described here may include features that are not included in all specific embodiments. A particular embodiment may include only a subset of all of the features described, or a particular embodiment may include all of the features described.

The leading digit(s) of reference numbers appearing in the Figures generally corresponds to the Figure number in which that component is first introduced, such that the same reference number is used throughout to refer to an identical component which appears in multiple Figures. Signals and connections may be referred to by the same reference number or label, and the actual meaning will be clear from its use in the context of the description.

The present invention relates to providing a medical system assisting more efficient and safer performance of medical procedures for clearing a catheter blockage and/or flushing a patient's bladder. For example, a patient with an indwelling urinary catheter, such as a Foley catheter, may have the catheter remaining in place for an extended period of time, and occasionally sediment, blood colts or other debris blocks the normal flow of urine from the patient's bladder out the drain lumen to the urine-collection bag. When such a patient with a blocked catheter presents in an emergency room, it is urgent to get a source of saline forced into the drain lumen of the catheter towards the patient's bladder. With respect to catheter-flushing problems, when a patient has a blocked catheter, it is desirable to clamp the downstream portion of the drain lumen of the catheter, and force a solution such as normal saline into the catheter upstream towards the blockage. Unfortunately, using a regular syringe often provides insufficient volume for clearing blockages, thus requiring a series of multiple draws of saline into the syringe from a source of saline, each draw alternated with a syringe connection to the Luer lock on the catheter, an injection of the saline into the catheter towards the bladder from the syringe and a disconnection from the Luer lock, each of which introduces an additional possibility of introducing an infectious agent or leaking fluid onto the health-care provider or the patient.

Presumably, the fluid force and the dilution effect of the saline will wash out the debris and prevent bladder contamination, infection, and scarring. The more fluid that can be inserted into the catheter in a single connection operation, the less chance of infection and greater the likelihood of success in clearing blockages. A syringe can sometimes be used for a clearing particularly strong blockage or when suction is needed as well as flushing, but the pressure from a hand-squeezed bottle or bag of saline is usually sufficient to push out unwanted debris. Unfortunately, to obtain large volumes of saline using a syringe, there are multiple repetitions of draw, connect, inject, disconnect, resulting in a higher likelihood of contaminated fluid being inserted to the catheter, or fluid leakage splashing onto a health-care provider or the patient. This is undesirable as the risk of infection is heightened and there are undesirable effects of getting a patient wet (for example, a trauma patient). The leaked fluid will also soil laundry and require increased housekeeping services, using conventional methods of clearing catheter blockages. This is also an inconvenience for otherwise healthy patients that have an indwelling catheter for extended period of time at home to travel to a busy emergency room to obtain assistance to remove the catheter blockage. These exigent catheter-clearing operations impede the ability for doctors or nurses to treat other waiting patients. The multiple repetitions of draw, connect, inject, and disconnect decrease the incentive for a health-care provider to use an appropriately large volumes of saline to optimally dilute the contaminant.

The present invention provides the ability to deliver a much larger quantity of liquid when using just one or just a few connections to the Luer lock coupled to a catheter, thus reducing the risk of infection. In addition to sterile saline, a medicine (such as an antibiotic) or an anticancer substance can be added into the saline bottle or bag for irrigating and treating an internal organ of the patient, such as by instilling the liquid into the organ, then holding it there for 30 to 60 minutes, or other suitable period of time, and then flushing again.

The scope of present invention is not limited to embodiments with particular Luer-lock-type connectors and ports, as commonly understood. In the present disclosure, wherever the descriptor “Luer” appears, with a capital “L,” it is to be understood the invention is not limited to a particular brand or maker of mating devices and that the invention may be implemented in other embodiments that use other types of fluid ports that have the same or a substantially similar function, which is to have a fluid port that is closed to prevent fluid flow through the interface when no connector device (such as syringe or the fluid interface 101 that includes a Luer-lock connector 110 of the present invention) is coupled to the port, but then automatically open to allow fluid flow through the port upon connection to such a connector. Similarly, the scope of present invention is not limited to embodiments with particular Foley-type catheters, as that term is commonly understood.

Certain catheters, such as indwelling Foley urinary catheters having two or three lumens in a common tube-shaped casing, can have the urine-drain lumen occasionally become blocked by sediment, blood clots, or the like. The drain lumen is typically connected to a urine collection bag using drain tubing that includes a side port receiver having a built-in Luer lock.

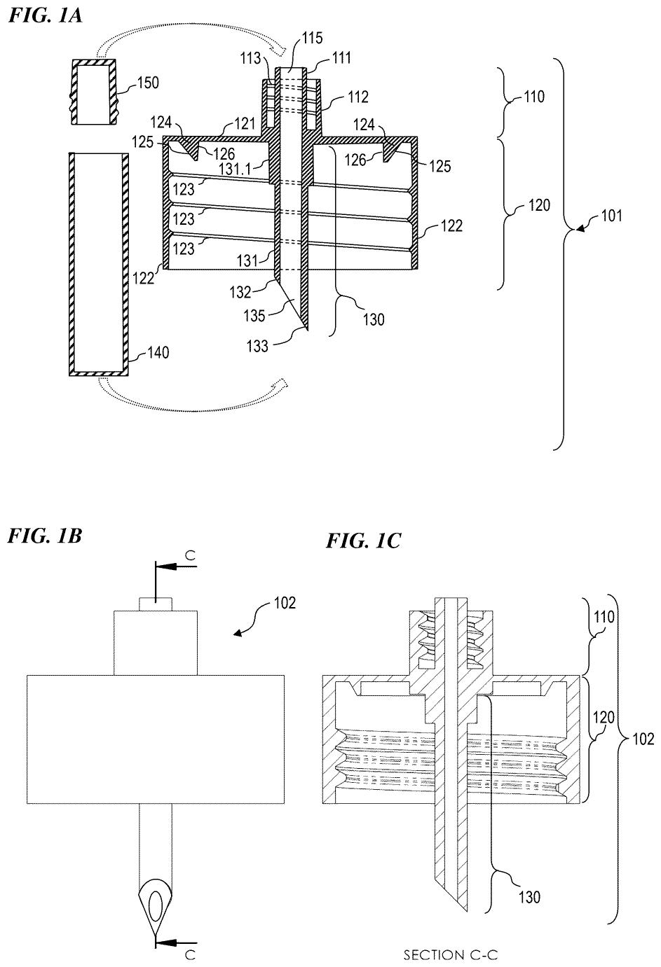

FIG. 1A is a cross-section view of a fluid interface system 101 that includes a Luer-lock connector 110, a threaded bottle connector 120 and a spike connector 130, along with a removable protective spike cover 140 and a removable protective Luer cover 150, according to some embodiments of the present invention.

In some embodiments, Luer-lock connector 110 is configured to connect to a receiver portion of a Luer lock, and includes a male central protrusion 111 configured to force the Luer lock into an “open” fluid-passing position when threads 113, located on the inside of walls 112, are screwed onto the Luer-lock receiver of tubing that runs from the drain lumen of the catheter to a urine-collection bag. The male central protrusion 111 has a longitudinal lumen 115 extending from Luer-lock connector 110 into the fluid of fluid source that is coupled to threaded bottle connector 120 and/or to spike connector 130.

In some embodiments, threaded bottle connector 120 includes a top wall 121, a cylindrical side wall 122 having internal threads 123 configured to engage with corresponding threads on a conventional polymer bottle that holds a sterile fluid such as saline. On the inside of top wall 121, some embodiments include a truncated conical structure 124 having a sloped conical outer surface 125 for engaging the inside of the top resilient lip of the polymer bottle to form a secure seal against fluid leaks, and optionally a cylindrical inner surface 126.

In some embodiments, spike connector 130 includes an outer wall 131, having a somewhat larger diameter base portion 131.1 for engaging the proximal end of engaging protective cover 140, and a tapered distal end 132 to central lumen 135 that extends to lumen 115 of Luer-lock connector 110, wherein distal end 132 has a sharp tip 133. Other embodiments include a sharp central tip and one or more side ports to central lumen 135 that extends to lumen 115 of Luer-lock connector 110. Yet other embodiments include both an end port and one or more side ports for fluid to enter lumen 135. Spike connector 130 is configured to pierce the piercing membrane or barrier of a spike port at the lower end of a saline bag or other flexible bag holding sterile fluid used to flush the blockage of an indwelling foley catheter. The piercing membrane of the saline bag, when closed, seals and prevents fluid from exiting the saline bag. Once spike connector has pierced the piercing membrane of the spike port on the saline bag, the user can squeeze the saline bag to force fluid into the blocked catheter to clear the blockage.

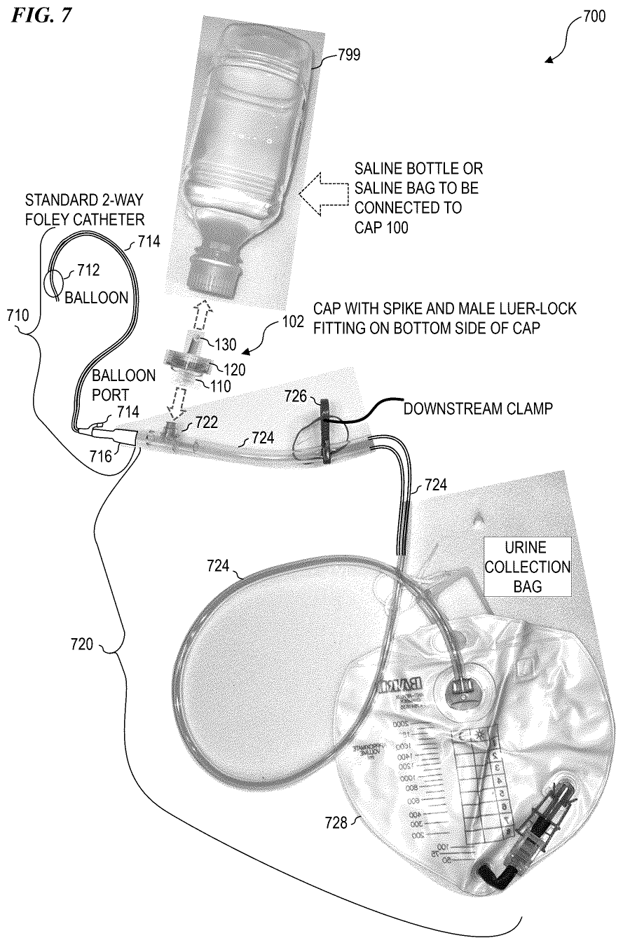

When in use (as shown in FIG. 7 described below), the Luer-lock connector 110 should be oriented in a generally downward direction to avoid possibly injecting air from the fluid source into the catheter. The tubing downstream from the Luer-lock interface should be clamped such that liquid forced through the Luer lock is directed upstream towards the catheter blockage. The fluid source is chosen to be squeezable by the person performing the procedure, such that when the fluid source is squeezed, liquid is forced through the Luer lock upstream towards the catheter blockage.

If a three-lumen Foley catheter is being used, the third lumen may be utilized to drain excess urine or other fluid as the injected liquid is going upstream in another lumen to clear the blockage.

FIG. 1B is a side view of another fluid interface 102, having slightly different proportions than fluid interface 101.

FIG. 1C is a cross-section view of fluid interface 102 (along section plane C of FIG. 1B) that includes a Luer-lock connector 110, a threaded bottle connector 120 and a spike connector 130, along with a removable protective spike cover 140 (as shown in FIG. 1A) and a removable protective Luer cover 150 (as shown in FIG. 1A), according to some embodiments of the present invention. Certain proportions of fluid interface 102 are slightly different than the corresponding features of fluid interface system 101, but the function and features are substantially similar to those described above.

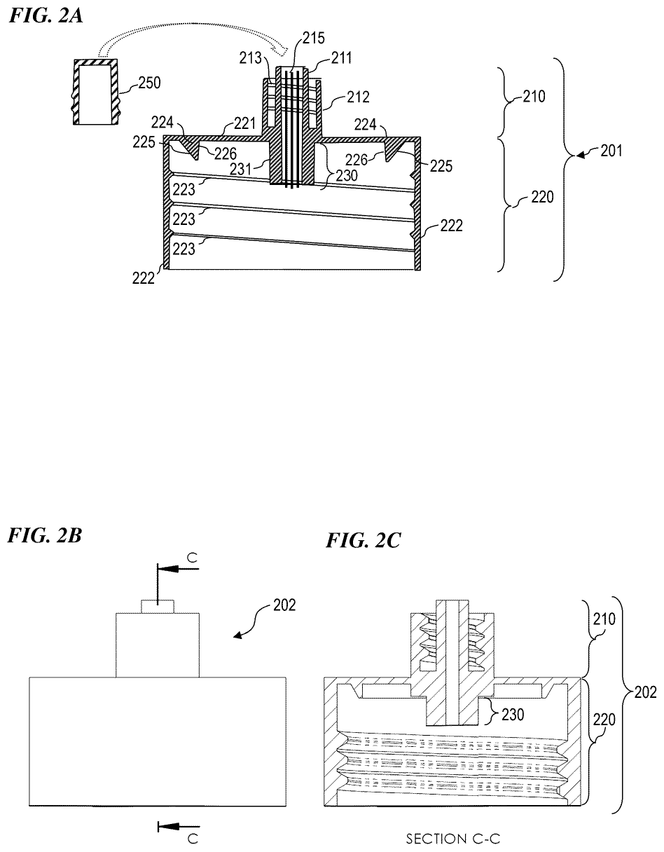

FIG. 2A is a cross-section view of a fluid interface 201 that includes a Luer-lock connector 210, a threaded bottle connector 220, along with a removable protective Luer cover 250, according to some embodiments of the present invention. In some embodiments, fluid interface 201 is substantially the same as fluid interface 101, but omits the spike connector.

In some embodiments, Luer-lock connector 210 is configured to connect to a receiver portion of a Luer lock, and includes a male central protrusion 211 configured to force the Luer lock into an “open,” fluid-passing, position when threads 213, located on the inside of walls 212, are screwed onto the Luer-lock receiver of the tubing that runs from the drain lumen of the Foley catheter to a urine-collection bag. The male central protrusion 211 has a longitudinal lumen 215 extending from Luer-lock connector 210 into the fluid of fluid source that is coupled to threaded bottle connector 220.

In some embodiments, threaded bottle connector 220 includes a top wall 221, a cylindrical side wall 222 having internal threads 223 configured to engage with corresponding threads on a conventional polymer bottle that holds a sterile fluid such as saline. On the inside of top wall 221, some embodiments include a truncated conical structure 224 having a sloped conical outer surface 225 for engaging the inside of the top resilient lip of the polymer bottle to form a secure seal against fluid leaks, and optionally conical structure 224 has a cylindrical inner surface 226.

FIG. 2B is a side view of another fluid interface 202, having slightly different proportions than fluid interface 201.

FIG. 2C is a cross-section view of fluid interface 202 along section plane C of FIG. 2B) that includes a Luer-lock connector 210, a threaded bottle connector 220 but no spike connector, according to some embodiments of the present invention. Certain proportions of fluid interface 202 as slightly different than the corresponding features of fluid interface system 201, but the function and features are substantially similar to those described above.

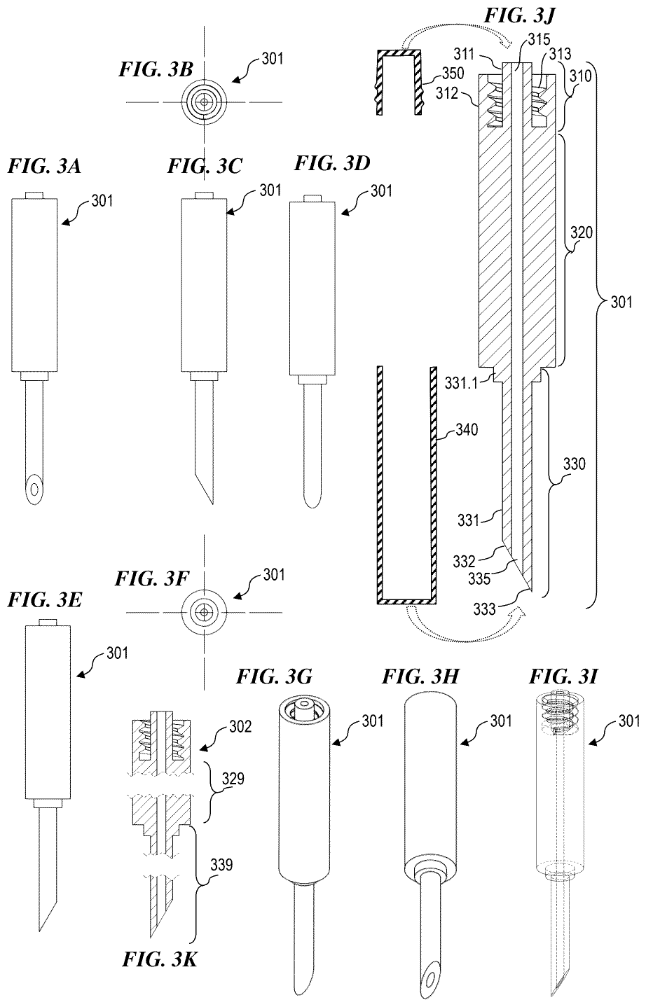

FIG. 3A is a front view of a fluid interface 301 that has a Luer-lock connector and a spike connector for interfacing with a bag of sterile fluid.

FIG. 3B is a top view of fluid interface 301.

FIG. 3C is a left-side view of fluid interface 301.

FIG. 3D is a back view of fluid interface 301.

FIG. 3E is a right-side view of fluid interface 301.

FIG. 3F is a bottom view of fluid interface 301.

FIG. 3G is an upper isometric view of fluid interface 301.

FIG. 3H is a lower isometric view of fluid interface 301.

FIG. 3I is a lower isometric view of fluid interface 301 showing internal structures in dashed lines.

FIG. 3J is a cross-section view of fluid interface 301 that includes a Luer-lock connector 310, a middle section 320 and a spike connector 330, along with a removable protective spike cover 340 and a removable protective Luer cover 350, according to some embodiments of the present invention.

In some embodiments, Luer-lock connector 310 is configured to connect to a receiver portion of a Luer lock, and includes a male central protrusion 311 configured to force the Luer lock into an “open” fluid-passing position when threads 313, located on the inside of walls 312, are screwed onto the Luer-lock receiver of tubing that runs from the drain lumen of the catheter to a urine-collection bag. The male central protrusion 311 has a longitudinal lumen 315 extending from Luer-lock connector 310 into the fluid of fluid source that is coupled to spike connector 330.

In some embodiments, middle section 320 provides a structure for the user to grasp when inserting spike connector 330 into a saline bag such that the Luer lock 310 and spike connector 330 do not get contaminated by touching those parts.

In some embodiments, spike connector 330 includes an outer wall 331, having a somewhat larger diameter base portion 331.1 for engaging the proximal end of engaging protective cover 340, and a tapered distal end 332 to central lumen 335 that extends to lumen 315 of Luer-lock connector 310, wherein distal end 332 has a sharp tip 333. Other embodiments include a sharp central tip and one or more side ports to central lumen 335 that extends to lumen 315 of Luer-lock connector 310. Yet other embodiments include both an end port and one or more side ports for fluid to enter lumen 335. Spike connector 330 is configured to pierce the piercing membrane or barrier of a spike port at the lower end of a saline bag or other flexible bag holding sterile fluid used to flush the blockage of an indwelling foley catheter. The piercing membrane of the saline bag, when closed, seals and prevents fluid from exiting the saline bag. Once spike connector 330 has pierced the piercing membrane of the spike port on the saline bag, the user can squeeze the saline bag to force fluid into the blocked catheter to clear the blockage.

FIG. 3K is a cross-section view of another fluid interface 302 that includes a Luer-lock connector 310, a middle section 329 of arbitrary length and a spike connector 339 of arbitrary length, according to some embodiments of the present invention.

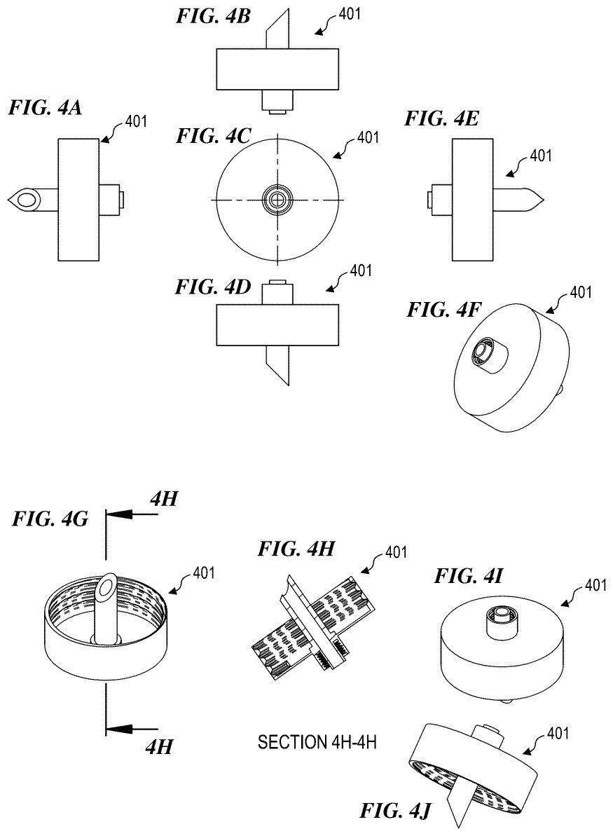

FIG. 4A is a front view of a fluid interface 401 that has a Luer-lock connector and a spike connector for interfacing with a bag of sterile fluid.

FIG. 4B is a left-side view of fluid interface 401.

FIG. 4C is a top view of fluid interface 401.

FIG. 4D is a right-side view of fluid interface 401.

FIG. 4E is a back view of fluid interface 401.

FIG. 4F is an upper isometric view of fluid interface 401.

FIG. 4G is a lower isometric view of fluid interface 401.

FIG. 4H is a cross-section view of fluid interface 401.

FIG. 4I is another upper isometric view of fluid interface 401.

FIG. 4J another lower isometric view of fluid interface 401.

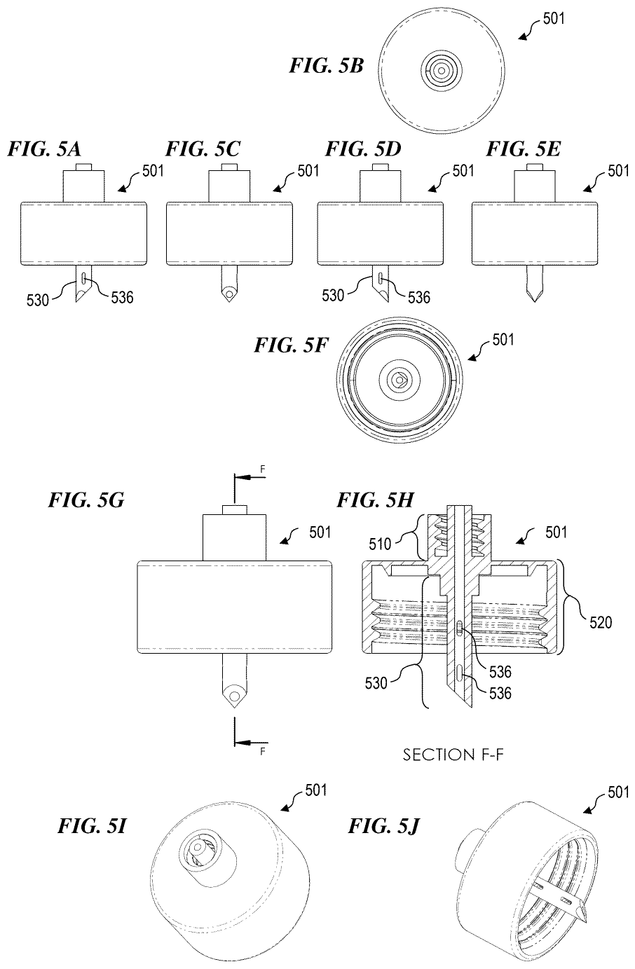

FIG. 5A is a right-side view of a fluid interface 501 that includes one or more side slots or through holes 536 in spike connector 530. Other aspects of fluid interface 501 are substantially similar to fluid interface 101, fluid interface 401, or fluid interface 601.

FIG. 5B is a top view of fluid interface 501.

FIG. 5C is a front view of fluid interface 501.

FIG. 5D is a left-side view of fluid interface 501.

FIG. 5E is a back view of fluid interface 501.

FIG. 5F is a bottom view of fluid interface 501.

FIG. 5G is a front view of fluid interface 501.

FIG. 5H is a cross-section view of fluid interface 501 that includes a Luer-lock connector 510, a threaded bottle connector 520 having rounded outer edges, and a slotted spike connector 520, according to some embodiments of the present invention.

FIG. 5I is an upper isometric view of fluid interface 501.

FIG. 5J is a lower isometric view of fluid interface 501.

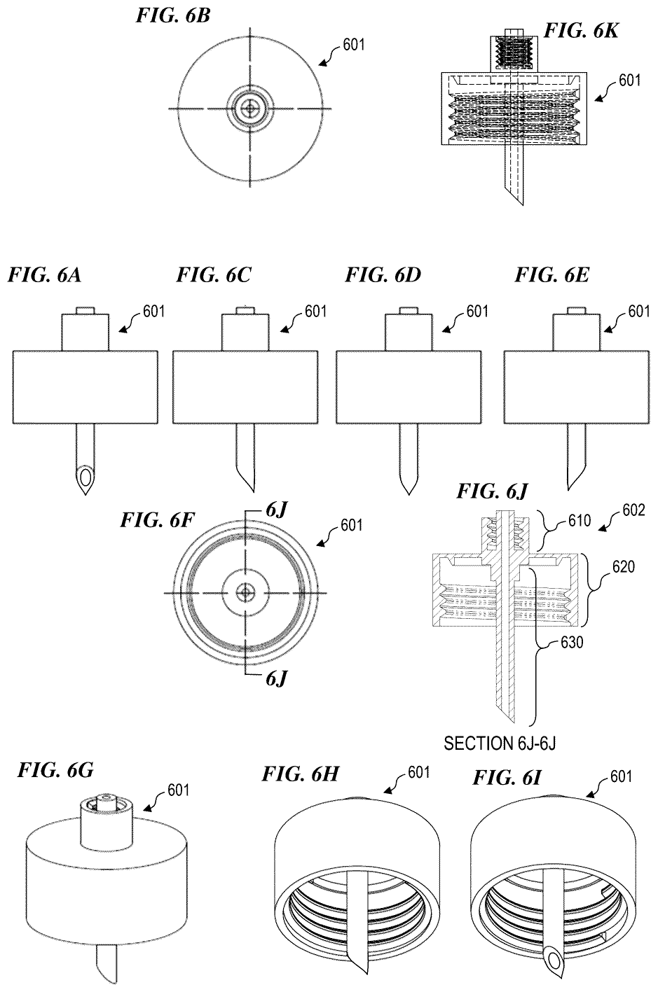

FIG. 6A is a right-side view of a fluid interface 601.

FIG. 6B is a top view of fluid interface 601.

FIG. 6C is a front view of fluid interface 601.

FIG. 6D is a left-side view of fluid interface 601.

FIG. 6E is a back view of fluid interface 601.

FIG. 6F is a bottom view of fluid interface 601.

FIG. 6G is an upper isometric view of fluid interface 601.

FIG. 6H is a lower isometric view of fluid interface 601.

FIG. 6I is another lower isometric view of fluid interface 601.

FIG. 6J is a cross-section view of another fluid interface 602 that includes a Luer-lock connector 610, a threaded cap (bottle connector) 620 and a spike connector 630, according to some embodiments of the present invention.

FIG. 6K is a side view of fluid interface 601 showing internal structures in dashed lines.

FIG. 7 is a block diagram side view of a system 700 for clearing catheter blockages in a foley catheter 710 and/or irrigating and flushing (such as flushing an organ with a medication, cancer-treatment fluid, or the like, or instilling medication, cancer-treatment fluid, or the like into the organ) a patient's bladder through foley catheter 710. Foley catheter 710 includes a balloon structure 712 on the distil end of tubing 714, wherein balloon structure 712, once filled with saline using balloon port 714, expands and holds the catheter in place in the patient's bladder. Urine drain system 720 includes a male connector end that is inserted into drain port 716 of foley catheter 710, a Luer-lock interface or port 722 having a Luer lock that remains closed unless and until a male Luer-lock connector (such as portion 110 of fluid-interface 102) is screwed on, drain tubing 724 that drains urine into urine-collection bag 728. A downstream clamp 726 is used to seal off the tubing to bag 728, for instances such as when it is desired to clear a blockage in foley catheter 710.

To clear a blockage in foley catheter 710, the cap on saline bottle 799 is removed and instead fluid interface 102 (with its protective spile cap 140 removed) is screwed onto saline bottle 799. Then the protective Luer-lock-connector cap 150 is removed, and Luer-lock connector 110 is screwed onto Luer-lock port 722, preferably with the Luer-lock connector 110 pointing in a generally downward direction such that fluid (such as saline), rather than air, is inserted into the drain port of foley catheter 710. Then, the health-care provider, or the patient herself or himself, squeezes the fluid source (saline bottle 799 or a saline bag, as the case may be) until it is sensed that the blockage is cleared, and the fluid interface 102 and its connected saline bottle are unscrewed from the Luer-lock port, and clamp 726 is release to drain the injected fluid and remaining urine into collection bag 728. If need be, clamp 726 may be again applied and a syringe may be then connected to Luer-lock port 722 to suck fluid from foley catheter 710, until a suitable flow path for urine is established.

In other embodiments, various features of FIG. 1A through 7 may be mixed, matched, added or omitted.

In some embodiments, the present invention provides a system for flushing a catheter that has, or is connected to, a Luer-lock connector. This system includes a monolithic structure that includes: a fluid-source connector configured to removably connect to a fluid source; a Luer-lock connector configured to operatively connect to the Luer lock on the catheter; and a fluid passageway between the fluid-source connector and the Luer-lock connector.

In some embodiments of the system, the fluid-source connector and the Luer-lock connector are integrally formed from a unitary rigid piece of polymer.

In some embodiments of the system, the fluid-source connector includes a saline-bag spike.

In some embodiments of the system, the fluid-source connector includes a female threaded saline-bottle connector.

In some embodiments of the system, the fluid-source connector includes both a saline-bag spike and a female threaded saline-bottle connector that surrounds at least a portion of the saline-bag spike. In some such embodiments, the saline-bag spike and the female threaded saline-bottle connector and the Luer connector are integrally formed from a unitary rigid piece of polymer. In some such embodiments, the system further includes a removable sheath for covering the spike.

In some embodiments of the system, the catheter is an indwelling Foley catheter that includes a first lumen for draining urine, and a second lumen for inflating a distal balloon tip with sterile water to keep the distal tip of the Foley catheter in place in a patient's bladder, wherein the Luer lock is operably coupled to the first lumen.

In some embodiments of the system, the fluid source contains saline.

In some embodiments of the system, the catheter is an indwelling foley catheter configured to drain urine from a patient's bladder.

In other embodiments, the present invention provides an apparatus that includes: a fluid-source connector; a Luer-lock connector configured to operatively connect to a Luer lock on an indwelling catheter; and a fluid passageway between the fluid-source connector and the Luer-lock connector, wherein the fluid-source connector and the Luer-lock connector are integrally formed from a unitary rigid piece of polymer. In some embodiments of the apparatus, the fluid-source connector includes a saline-bag spike. In some embodiments of the apparatus, the fluid-source connector includes a female threaded saline-bottle connector. In some embodiments of the apparatus, the fluid-source connector includes both a saline-bag spike and a female threaded saline-bottle connector that surrounds the saline-bag spike. In some embodiments of the apparatus, the indwelling catheter is an indwelling Foley catheter that includes a first lumen for draining urine and a second lumen for inflating a distal balloon tip with sterile water to keep the distal tip of the Foley catheter in place in a patient's bladder, and wherein the Luer lock is operably coupled to the first lumen

In yet other embodiments, the present invention provides a Luer-connection apparatus that includes: a housing including a first side wall having an inside surface defining a lumen with a central axis, a proximal end, and a distal end including a male Luer tip having a passageway therethrough providing fluid communication between the proximal end and the distal end; and a fluid-source connector configured as part of the housing to connect to a fluid source that has squeezable side walls, wherein the proximal end of the lumen is open inside the fluid source when the fluid source is affixed to the fluid-source connector.

In some embodiments of the Luer-connection apparatus, the fluid-source connector includes a saline-bag spike.

In some embodiments of the Luer-connection apparatus, the fluid-source connector includes a female threaded saline-bottle connector.

In some embodiments of the Luer-connection apparatus, the fluid-source connector includes both a saline-bag spike and a female threaded saline-bottle connector that surrounds the saline-bag spike.

In some embodiments of the Luer-connection apparatus, the male Luer tip is configured to connect to a Luer lock that remains closed unless and until the male Luer tip is screwed onto the Luer lock.

It is to be understood that the above description is intended to be illustrative, and not restrictive. Although numerous characteristics and advantages of various embodiments as described herein have been set forth in the foregoing description, together with details of the structure and function of various embodiments, many other embodiments and changes to details will be apparent to those of skill in the art upon reviewing the above description. The scope of the invention should be, therefore, determined with reference to the appended claims, along with the full scope of equivalents to which such claims are entitled. In the appended claims, the terms “including” and “in which” are used as the plain-English equivalents of the respective terms “comprising” and “wherein,” respectively. Moreover, the terms “first,” “second,” and “third,” etc., are used merely as labels, and are not intended to impose numerical requirements on their objects.

What is claimed is:

1. A system for flushing fluid through a catheter coupled to a Luer-lock interface having a Luer lock, wherein the Luer lock opens when a male Luer connector is connected to the Luer-lock interface, the system comprising:
a monolithic structure that includes:
a fluid-source connector configured to removably connect to a fluid source;
a male Luer connector configured to operatively connect to the Luer-lock interface on the catheter; and
a fluid passageway between the fluid-source connector and the Luer connector.

2. The system of claim 1, wherein the fluid-source connector and the Luer connector are integrally formed from a unitary rigid piece of polymer.

3. The system of claim 2, wherein the fluid-source connector includes a saline-bag spike.

4. The system of claim 3, further comprising a removable sheath for covering the spike.

5. The system of claim 2, wherein the fluid-source connector includes a female threaded saline-bottle connector.

6. The system of claim 1, wherein the fluid-source connector includes both a saline-bag spike and a female threaded saline-bottle connector that surrounds at least a portion of the saline-bag spike.

7. The system of claim 6, wherein the saline-bag spike and the female threaded saline-bottle connector and the Luer connector are integrally formed from a unitary rigid piece of polymer.

8. The system of claim 7, further comprising a removable sheath for covering the spike.

9. The system of claim 1, wherein the fluid source contains sterile saline.

10. The system of claim 1, wherein the catheter is an indwelling Foley catheter that includes a first lumen for draining urine and a second lumen for inflating a distal balloon tip with sterile water to keep the distal tip of the Foley catheter in place in a patient's bladder, and wherein the Luer lock is operably coupled to the first lumen.

11. An apparatus comprising:
a fluid-source connector;
a Luer-lock connector configured to operatively connect to a Luer lock on an indwelling catheter; and
a fluid passageway between the fluid-source connector and the Luer-lock connector, wherein the fluid-source connector and the Luer-lock connector are integrally formed from a unitary rigid piece of polymer.

12. The apparatus of claim 11, wherein the fluid-source connector includes a saline-bag spike.

13. The apparatus of claim 11, wherein the fluid-source connector includes a female threaded saline-bottle connector.

14. The apparatus of claim 11, wherein the fluid-source connector includes both a saline-bag spike and a female threaded saline-bottle connector that surrounds the saline-bag spike.

15. The apparatus of claim 11, wherein the indwelling catheter is an indwelling Foley catheter that includes a first lumen for draining urine and a second lumen for inflating a distal balloon tip with sterile water to keep the distal tip of the Foley catheter in place in a patient's bladder, and wherein the Luer lock is operably coupled to the first lumen.

16. A Luer-connection apparatus comprising:
a housing including a first side wall having an inside surface defining a lumen with a central axis, a proximal end, and a distal end including a male Luer tip having a passageway therethrough providing fluid communication between the proximal end and the distal end; and
a fluid-source connector configured as part of the housing to connect to a fluid source that has squeezable side walls, wherein the proximal end of the lumen is open inside the fluid source when the fluid source is affixed to the fluid-source connector.

17. The Luer-connection apparatus of claim 16, wherein the fluid-source connector includes a saline-bag spike.

18. The Luer-connection apparatus of claim 16, wherein the fluid-source connector includes a female threaded saline-bottle connector.

19. The Luer-connection apparatus of claim 16, wherein the fluid-source connector includes both a saline-bag spike and a female threaded saline-bottle connector that surrounds the saline-bag spike.

20. The Luer-connection apparatus of claim 16, wherein the male Luer tip is configured to connect to a Luer lock that remains closed unless and until the male Luer tip is screwed onto the Luer lock.
